(19) United States
(12) Patent Application Publication
(10) Pub. No.: US 2020/0229361 A1
(43) Pub. Date: Jul. 23, 2020

(54) Method and System of Determining Soil-Water Properties

(71) Applicant: James Canyon, San Diego, CA (US)

(72) Inventor: James Canyon, San Diego, CA (US)

(21) Appl. No.: 16/253,152

(22) Filed: Jan. 21, 2019

(51) Int. Cl. A01G 25/16; G01N 33/24; G01B 11/24

(52) U.S. Cl. CPC A01G 25/167; G01N 27/223; G01B 11/24; G01N 33/246

(57) ABSTRACT

A system and method for simulating soil moisture of farmland and large agricultural areas in three dimensions is described. The invention utilizes numerical techniques to solve a three dimensional boundary value problem which is defined by the soil to air interface (surface, x and y), and soil below surface (depth along the z axis) over the area of interest. Solved are key parameters which describe the soil which include soil particle size distribution (soil type), Hydraulic conductivity (water flow in the Z axis) Soil water diffusivity (water moving in the x, and y direction). This model will result in delivering soil moisture readings and soil water storage as a function of time thereby helping local managers of farmland or large agricultural areas to optimize watering and care of crops.

### RELATED APPLICATIONS

This application claims priority to U.S. provisional application No. 62/619,765, filed Jan. 20, 2018 and entitled “Method and system of simulating soil-water properties,” which is incorporated herein in its entirety. This application is also a continuation in part to U.S. patent application Ser. No. 16/035,612, filed Jul. 14, 2018, which is a continuation of U.S. patent application Ser. No. 15/057,885, filed Mar. 1, 2016, now U.S. Pat. No. 10,028,425, entitled “System, Apparatus, and Method for Remote Soil Moisture Measurement and Control,” which claims priority to U.S. provisional application 62/127,243, filed Mar. 2, 2015, both of which are incorporated herein by reference.

### FIELD OF THE INVENTION

The present invention relates to a method and device that measures soil moisture using active radar and determines soil moisture in using a three-dimensional boundary analysis.

### BACKGROUND

Most of the world is suffering in a chronic state lacking fresh drinking water. This leads to a shortage of water for agriculture, which makes it expensive or impossible to grow crops effectively. Increased need for water conservation in recent years has led to higher food prices and higher costs for farmers and consumers alike. The need for conservation has stemmed from higher demands on food production and higher population bases in localized areas. Water authorities around the United States, and the world are enacting watering limits and water usage expectations to ensure the valuable resource is being used carefully. In addition to agricultural needs, residential, sporting and landscaping all consume water at an alarming rate. It has been shown that in commercial crops, the amount of water used will greatly affect the profitability of the farm and therefore farmers are economically motivated to use the water carefully.

It would be desirable therefore to have an innovative sensor technology such that an accurate watering and fertilizing regime can be constructed to optimize water use and minimize over fertilization runoff. Large areas can be monitored and optimized at extremely low costs utilizing proposed remote sensing technologies described herein, thereby improving the production of food and other agricultural products. Since it is clear that water conservation is important for society, this invention describes a method and apparatus to be able to enable optimal water and fertilizer usage for a given landscape or crop. The subject of this invention is to, for a given crop or landscape, enable the water user to reduce the water usage to the optimal point and therefore minimize the cost of water, and/or optimize the yield in the growing of commercial food crops.

In order to enable this ability several pieces of technology are necessary. Some of the technology has been developed and some of the technology is the subject of this invention. In order to optimize cost further, technology choices were made to enable the optimal cost structure. Other choices could yield similar results in terms of water usage and therefore could still result in significant savings for the user, however they would not yield the ideal cost savings.

With reference to FIG. 1, well established soil properties are summarized below.

Soil Properties.

Soil is made up of various percentages of sand, silt, and clay as shown in FIG. 1. The ability for soil to retain water is highly dependent on the average particle size as the water “takes up the space” between the soil particles and the water tension is the mechanism which holds it in position. Many commercial farms or large agricultural areas do not have a uniform soil type consequently various areas require more/less water to maintain the same crop yield and quality.

Water Movement Through Soil.

Soil-water flux, “J” is defined as the quantity of water leaving the profile per unit time across a specific depth and is equal to the −1*hydraulic conductivity “K(θ)”, where θ is the volumetric water concentration in the soil, multiplied by the head or “dH”, or J=−K(θ)*dH. Combining Soil-water flux with the equation of continuity yields a differential equation solution to hydraulic conductivity or dθ/dt=d/dz(K(θ)*(dH/dz)) which is a measurement of soil water flow in the z direction.

The head, H, is affected by several factors. Most important are gravity and the tension in the soil. The latter is a strongly nonlinear function of θ, as is the conductivity, K(θ). As a result, the differential equation describing water movement is strongly nonlinear, making it tricky to solve successfully using numerical techniques. The most common technique is simple integration by forward or (usually) reverse differencing. While simple to implement, these methods sometimes fail to yield a solution for many kinds of problems. Another technique, which has been used in other technologies to solve multivariate nonlinear systems of equations, is the multidimensional form of Newton's method.

### SUMMARY OF THE INVENTION

A system and method for simulating soil moisture of farmland and large agricultural areas in three dimensions is described. The invention utilizes numerical techniques to solve a three dimensional boundary value problem which is defined by the soil to air interface (surface, x and y), and soil below surface (depth along the z axis) over the area of interest. Solved are key parameters which describe the soil which include soil particle size distribution (soil type), Hydraulic conductivity (water flow in the Z axis) Soil water diffusivity (water moving in the x, and y direction). This model will result in delivering soil moisture readings and soil water storage as a function of time thereby helping local managers of farmland or large agricultural areas to optimize watering and care of crops. Furthermore scenarios can be fed into the model which will further add in its value to local managers as they will be able to test different scenarios (crop type, crop maturity, weather conditions, water conditions, etc).

Prior to building a water transport through soil model one must measure the soil type and soil moisture as a function of discrete time for a single volume then use this knowhow to scan a two dimensional surface of these volumes in order to calculate diffusivity (lateral water movement). Soil type and soil moisture as a function of time are generated by successive scans of the farmland in question. For simplicity we will discuss this process in 4 steps. Determination of soil type, Determination of velocity of propagation in the soil, Determination of soil moisture as a function of depth, and lastly generation of a water transport through soil model as described. It is to be noted that all measurements are limited to the resolution of the system. The system resolution is a strong function of radar cross section hitting the ground to determine X-Y, radar frequency determines depth of penetration, Radar bandwidth determines resolution in Z.

Simplified Measurement of Soil Type:

Referring to FIG. 4, FIG. 300 a patch of soil or a pixel as determined by the system resolution in XYZ as seen by radar can be thought of as having two major reflections, the reflection between the air/ground interface and the reflection generated by the discontinuity of the boundary between the unsaturated region (Er1) and the saturated region Er2. Utilizing a radar modulation which converts reflected time of arrival to frequency or code (CDMA techniques) we determine soil type using successive scans. With each scan we calculate the rate of movement of the unsaturated (U)/saturated (S) region or dU/dS. A simplified model showing the unsaturated/saturated region move as a function of time is shown in 302,303,304. For clarity, we show an actual radar measurement of successive scans in 305. Utilizing this method, augmented with crop type, climate, and local irrigation quantities we easily generate a model of soil type as a function of depth for spot size defined by radar cross section XY and max radar depth Z, at resolution defined by radar bandwidth.

Calculation of Velocity of Propagation Along the Soil Depth Profile:

Velocity of propagation and propagation losses within the soil are required in order to accurately determine measurement depth. These parameters are a function of soil type (step 1) and are summarized in lookup tables for the three types of soil Coarse (Sandy) Medium (Sandy Loam), and Fine (Clay). Referring to FIG. 400 I have included plots of the real and imaginary parts of dielectric constant for each type of soil. Velocity of propagation is a function of soil type, and dielectric constant and is simply expressed as Vp=1/SQRT (Er or K) where K may be a complex number to include losses within the dielectric from which soil conductivity is accounted for.

Calculation Soil Moisture in the Unsaturated Region:

For a given volume as defined by system parameters with a given measurement resolution in Z also defined by system parameters we are free to calculate soil moisture as a function of depth. Referring to FIG. 6, FIG. 500 we show the system defined volume with system defined resolution in Z. Referring to 502 To this volume a modulated radar waveform is incident with known incident power Pi is applied. The receiver receives the reflected power as a function of depth Pr at t0-t5. The transmitted power equals the sum of the reflected power minus losses or Pi=Pr(t0)+Pr(t1+11)+Pr(t2+12)+Pr(t3+13) . . . where 11, 12, 13 etc. include losses in the soil and losses due to multiple reflections. This equation is easily solved using a form of Newton-Raphson (NR) optimization, the equation can also be solved in closed form if the resolution steps in z are small.

Building a Water Transport Through Soil Model:

At this point we have a good idea of what is happening in a single pixel. We know soil type as a function of depth, have a model of Er of soil for each resolution step in Z (velocity of propagation), our model of Er includes losses within each step (soil conductivity), and lastly we have soil moisture as a function of depth. All this for a single pixel or spot on the ground which is good enough to develop a one dimensional water transport model. Volumetric water concentration as a function of time is now defined for the target pixel and its adjacent pixels along the volume. We next solve for hydraulic conductivity or dθ/dt=d/dz(K(θ)*(dH/dz)) which is a measurement of soil water flow in the z direction within a single pixel. To develop a three dimensional water transport model we scan the surface of a farm or greenspace and solve the above for each pixel in the surface then solve the hydraulic conductivity equation in three dimensions or dθ/dt=d/dv(K(θ)*(dH/dv))

Referring now to FIG. 2 and TO FIG. 3, examples of the preferred embodiment are described. FIG. 2 is a block diagram of a soil moisture sensor 100, and FIG. 2 is a block diagram of an irrigation control system 200. The subject of this invention starts with a mechanism designed to remotely measure soil moisture in a field or commercial Referring now to FIG. 2 and TO FIG. 3, examples of the preferred embodiment are described. FIG. 2 is a block diagram of a soil moisture sensor 100, and FIG. 2 is a block diagram of an irrigation control system 200. The subject of this invention starts with a mechanism designed to remotely measure soil moisture in a field or commercial landscape area such as a golf course. In farm and

The system described utilizes active radar which sends a modulated signal (at various frequencies as required) to the soil, the radar penetrates the soil at a depth inversely proportional to the transmitting frequency and a portion of the signal is reflected back to the transmitter based on the difference in dielectric constant discontinuities between air and soil and various soil types and moisture as the signal penetrates to the maximum depth. When the signal returns to the receiver, if the apparatus uses one antenna the transmitter is turned off, if using multiple antennas there is no need to turn off the transmitter. Knowing the transmit power and receive power, the device then calculates the reflection coefficient of the soil at various depths thereby determining the mean dielectric constant of the soil over a volume defined by radiation area as a function of depth. at that frequency and thereby determines the mean dielectric constant of the soil over a volume defined by radiation area and a depth which is a function of transmit frequency.

The modulated signal is modulated in such a way to optimize the measurement of receive power as a function of time, this allows the system to image dielectric constant as a function of depth (very well known in radar design, one method called chirped radar or FMCW radar converts round trip time of arrival to an offset frequency. Other methods such as code division covert roundtrip time of arrival to an offset code.)

By utilizing different discrete frequencies, for example, 400 MHz, 200 MHz, 100 MHz, 27 MHz. Soil moisture can be determined as a function of depth simply by measuring the reflection coefficient at different frequencies, for example, at 400 MHz we measure a depth of ˜4 inches, at 200 MHz we measure a depth of ˜8 inches therefore using simple math we can deduce soil moisture at 0-4 inches and at 4-8 inches depth. By utilizing different swept frequencies for example 200 MHz-400 MHZ and 450 MHz-1 GHz soil moisture can be determined as a function of depth utilizing a time of arrival to frequency transform as prescribed by FMCW radar techniques AND simultaneously avoid band between 400 and 450 MHz as described. Utilizing this swept frequency example, resolution is a function of 1/transmission bandwidth and dielectric constant of the soil.

The main advantage of using this technique is that measurements are made utilizing a radar that is passed above the soil without requiring direct contact with soil; installation of soil moisture sensors and systems are expensive and only provide soil moisture at one location these sensors are typically removed prior to harvesting making them time consuming. Another advantage to this technique is its ability to deliver very accurate measurements of soil moisture taken at regular or irregular intervals in x and y and z as described above.

A typical embodiment would be to mount the device into a flying device or ground robot which is either automated or driven/flown by hand, thus allowing 3d mapping of soil moisture whenever required by the agronomist or manager of the farm or open green area.

### BRIEF DESCRIPTION OF THE DRAWINGS

FIG. 1 shows a soil type diagram.

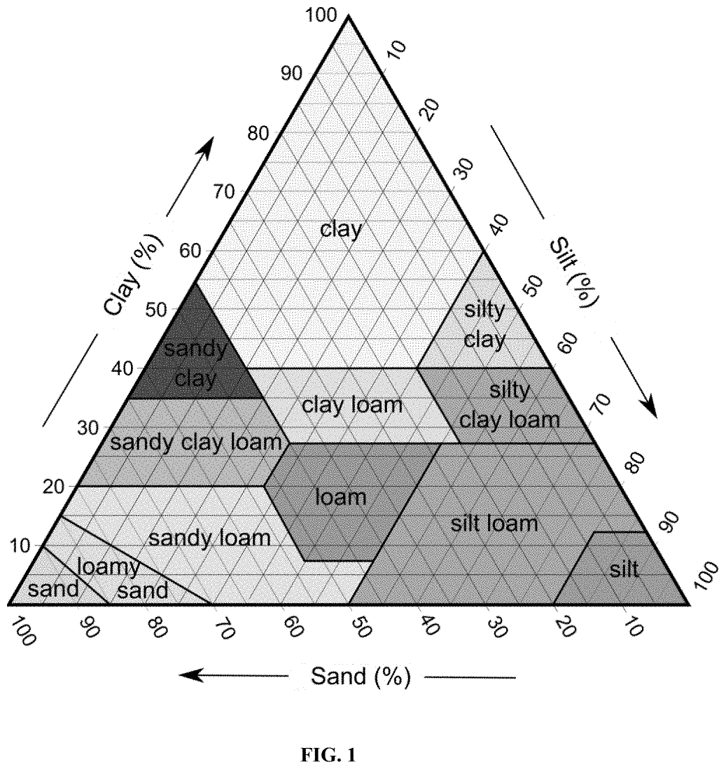

FIG. 2 is a block diagram of a soil moisture sensor in accordance with the present invention.

FIG. 3 is a block diagram of a soil moisture sensor system diagram in accordance with the present invention.

FIG. 4 is a block diagram of the boundary depth between the unsaturated region and saturated region of soil as a function of times, in this case three times t(0), t(1), t(2). Additionally picture 305 shows an example FMCW radar measurement showing this reflection interface move as a function of time (taken on 10/3, 10/9, 10/13 of 2018)

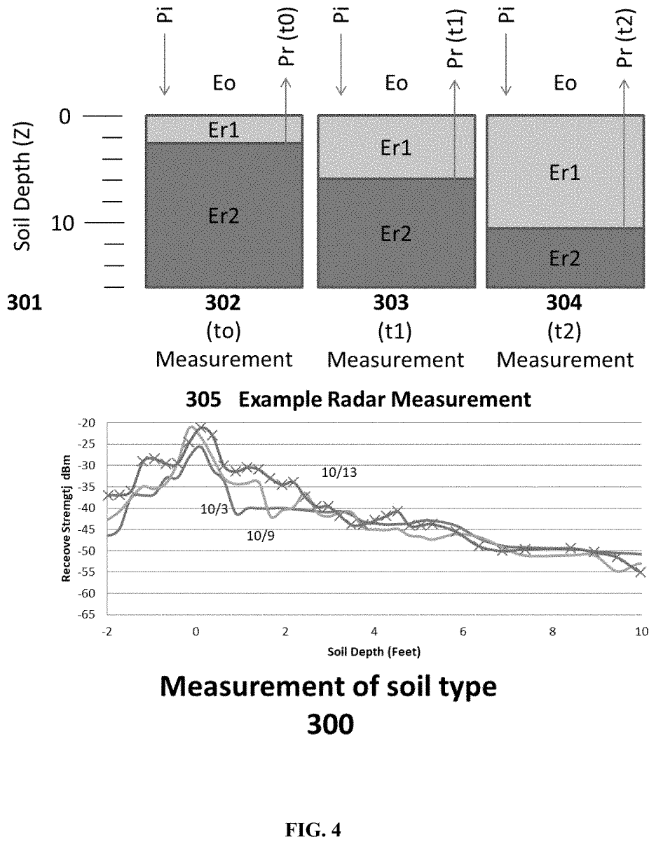

FIG. 5 are plots of dielectric constant vs volumetric soil water content for three types of soil. These curves are well known.

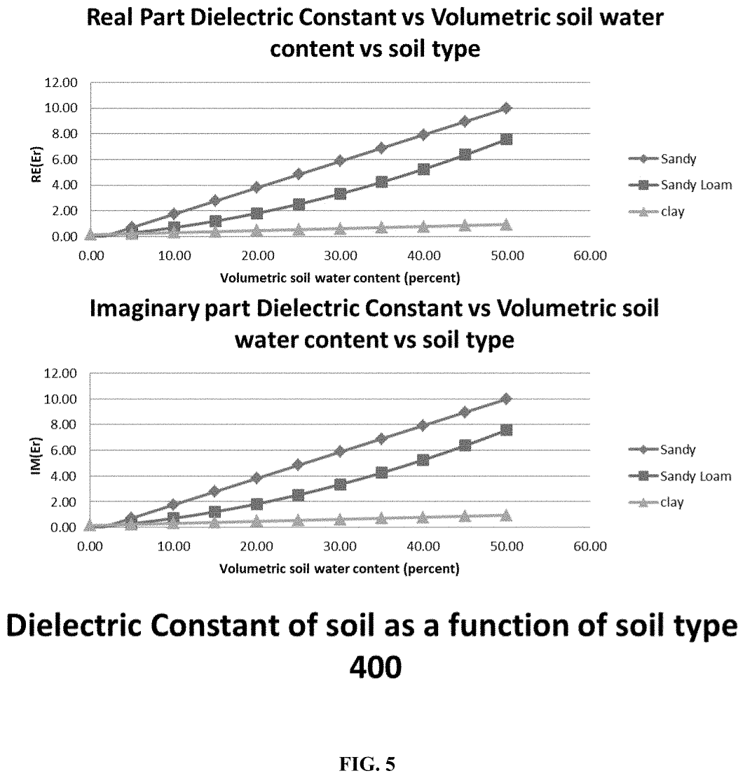

FIG. 6 shows one volume as defined by the radar cross section XY and the radar Depth Z and the received power resolution (Determined by the radar bandwidth). This shows the first reflections at each boundary interface equal to the radar resolution. Incident power is equal to the reflected power in this case (Sum of Pr from time 0 to time 5.

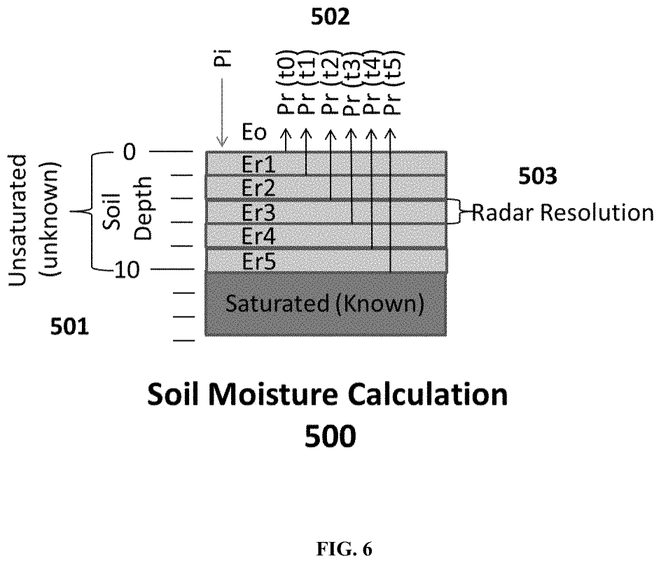

### DETAILED DESCRIPTION OF THE PREFERRED EMBODIMENT

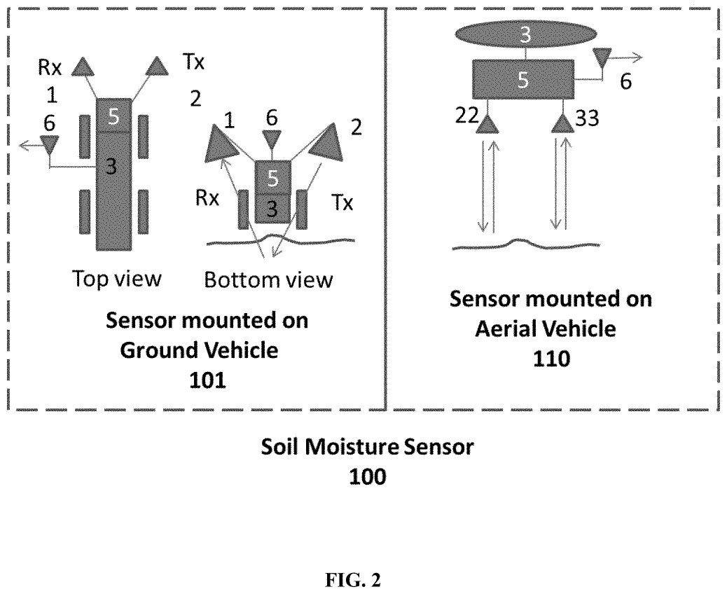

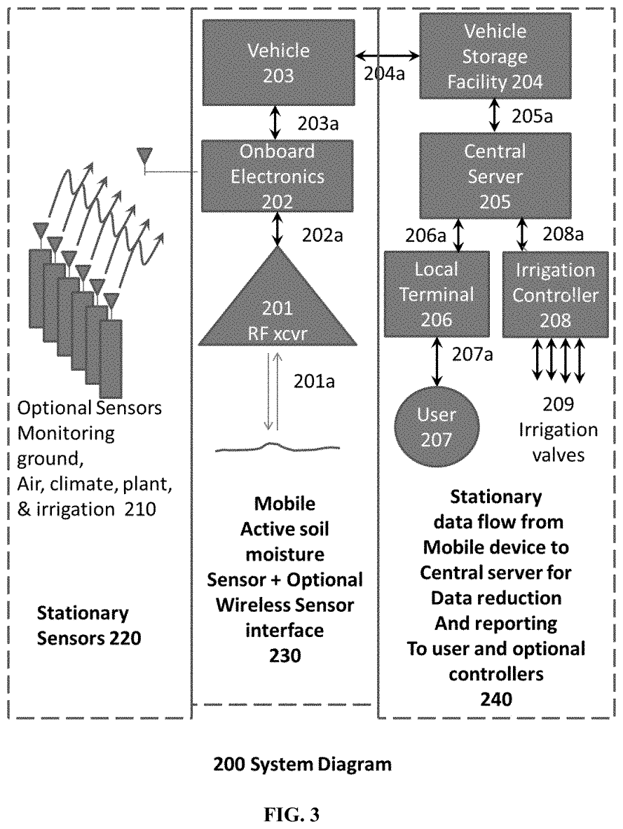

Referring now to FIG. 2 and TO FIG. 3, examples of the preferred embodiment are described. FIG. 2 is a block diagram of a soil moisture sensor 100, and FIG. 2 is a block diagram of an irrigation control system 200. The subject of this invention starts with a mechanism designed to remotely measure soil moisture in a field or commercial landscape area such as a golf course. In farm and large commercial landscape systems, implementation is provided by placing data collection electronics onto a ground vehicle FIG. 101 and sub FIG. 3 or aerial vehicle FIG. 110 and sub FIG. 3. Data collection is performed by electronics located in block 5 that I designed to transmit and receive radio frequency signals such that they hit the ground and are reflected to a receiver. For ground vehicles transmission antenna 2 and receive antenna 1 are separate. On aerial vehicles, transmission antennas and receive antennas 22 and 33 are either shared or not shared. Both ground and aerial vehicles utilize electronics 5 to create radio frequencies and measure the return radio frequency parameters. Parameters are stored in a server where algorithms are implemented to generate actionable data for use by the agronomist or farmer. Once actionable data is generated, it can be disseminated to the user via a computer or handheld internet connected device. If requested the actionable data can be delivered to a ground equipment controller with the capability to control devices on site.

One embodiment of the irrigation system is illustrated at 101. In this embodiment, a wireless transmitter 2 and wireless receiver 1 are employed to transmit electromagnetic energy into the ground and receive the reflected energy for processing. In the simple embodiment shown here, the wireless moisture receiver 1 can compare received power as referenced to the transmitted power and calculate soil moisture.

A second embodiment of the system is illustrated in FIG. 110. In this embodiment, a wireless transceiver 22 is employed to transmit electromagnetic energy into the ground and receive the reflected energy from the ground using the same antenna for processing. In the simple embodiment shown here, the wireless electronics connect the antenna using a switch from the transmitter to the receiver then calculate the effective reflection coefficient of the ground. A second antenna is shown 33. This antenna is tuned to a second frequency to improve soil moisture measurement as a function of depth. Both sets of data are then fed to a local controller 5 which determines where to make a measurement and when. The controller also stores the data for future download to the central computer via use of memory or wireless connection via transceiver 6.

An embodiment of the overall system design is illustrated in FIG. 200. The system design is shown as three major blocks (220, 230, 240) which cooperate and work together. Optional stationary sensors 220 which communicate with the mobile device, which is also capable of measuring soil moisture 230, and the vehicle storage facility and central server and interface to the user and irrigation valve interface 240.

Stationary sensors 220 are typically placed in fixed locations in the field of interest or to be used as reference values for calibration. These sensors can comprise the following but are not limited to the following: Soil moisture sensors, soil salinity and PH sensors, weather sensors, plant nitrogen sensors, water flow rate sensors, water pressure sensors water valves and could be other sensors or actuators which help with calibration of the system or control of irrigation or other parts of an individual farm or greenspace. These sensors may be wireless such that when the ground based or airborne mobile device comes within range of the sensor, data is transferred from the sensor to mobile device and vice versa. This allows the system to work over very large farming concerns while not burdening the sensors with transceivers capable of communicating over multiple miles or batteries capable of supporting these large transmit powers.

Mobile active soil moisture sensor and optional wireless sensor interface 230 is the heart of the system. This device is designed to move throughout the area of interest and both gather sensor data from 220 when the device is within wireless range and measure soil moisture utilizing the soil moisture sensor described in 100. The device is designed to move throughout the area of interest either by pre-program method or by manual method. During its data gathering task, the onboard electronics may either store all measurements or communicate the measurements as it measures/retrieves them or any portion thereof. Once the device has completed its course, it returns to the vehicle storage facility 204 and parks whereby a link either wired or wireless or combination 205 is used to download the data to a central server. The central server runs algorithms and reduces the data to a format which is usable by both the user 206 and 207 and data capable of delivery to a controller 208 and 209

Theory of Operation

The system works based on a few basic parameters surrounding electromagnetic wave propagation in air, electromagnetic wave reflection in a the presence of a dielectric discontinuity, the dielectric constant of air and the dielectric constant of soil varies as a function of how moist it is and lastly the penetration depth of an electromagnetic wave into a dielectric discontinuity.

Dielectric constant of soil. Some of the most expensive soil moisture measurement devices on the market today utilize something called time domain reflectometry. These devices are designed to measure the dielectric constant of soil. The dielectric constant of soil varies as a function of wet the soil is. The reason for this variation is the fact that dry soil like sand or loam has a dielectric constant of approximately 4. Water has a dielectric constant of approximately 18 times that of dry soil and therefore as water molecules mix with dry soil the dielectric constant changes from 4 to much greater than 50 depending on the soil type and moisture content.

Wave Propagation in the Presence of Mismatched Dielectrics.

When a wave that is traveling in air reaches a mismatched dielectric interface a portion of the power will continue through the transition and a portion of the wave will be reflected. The reflection is a function of the dielectric constant of air and the dielectric constant of the medium the wave comes in contact with. Specifically Er, the reflected wave is equal to the Ei the incident wave multiplied by the reflection coefficient at the interface, or n1—dielectric constant of air and n2 the dielectric constant of dry soil plus water.

Therefore if we know the dielectric constant of air, n1, the incident wave power Ei and can measure the reflected power Er then we can calculate the average dielectric constant of the soil n2.

Eᵣ=((n₁−n₂)/(n₁+n₂))*Eᵢ i.

Soil Moisture Calculation.

Calibration charts of dielectric constant as a function of soil moisture and soil type are common with companies that measure soil moisture using time domain reflectometry. Given Er, Ei, and n1, we can calculate n2, the dielectric constant of the soil and apply a calibrated lookup table dielectric constant and soil moisture to ultimately derive soil moisture.

Soil Moisture Measurement Maximum Depth.

When an electromagnetic wave hits the boundary of dielectric discontinuity the wave penetrates the dielectric proportionally to the wavelength of the transmitted signal and the dielectric constant mismatch. This penetration depth is similar to skin depth and is a complicated function which is dependent on, resistivity (sigma), dielectric constant (er), permittivity of soil (u) and 1/transmission frequency

$$δ = ( \frac{\sqrt{2}}{ω \sqrt{μɛ}} ) [ \sqrt{1 + {( \frac{σ}{ωɛ} )}^{2}} - 1 ] \text{?} \\ \text{?} \text{indicates text missing or illegible when filed} \qquad ( 1 )$$

In short, the depth of influence can be measured and each time the frequency is reduced the depth is increased. Therefore measuring at multiple frequencies is similar to measuring the soil moisture at different depths as defined by (1). This makes it simple to determine soil moisture as a function of depth.

Remote Sensing of Soil Moisture.

Soil moisture apparatus consists of a transmitted radio wave pointed at the soil to be tested. The incident wave travels at the speed of light and collides with the soil and penetrates the soil by a distance known as the skin depth it is this region that sets up the conditions for the reflected wave to radiate back to the receiver. This is also the depth that the sensor is averaging.

Therefore knowing the transmit power, antenna gain, antenna beam pattern, frequency, free space losses and scattering losses and measuring the receive power, the apparatus can easily measure reflected power and therefore can infer soil moisture.

The proper addition of modulation of the transmitted waveform and demodulation of the receive waveform to create a time of arrival to frequency conversion such as FMCW or a time of arrival to code conversion such as CDMA techniques allows improved resolution as a function of depth.

Modeling of Water Movement in Soil.

Movement of water in unsaturated soil is described by a nonlinear diffusion equation. That equation can be integrated in a number of ways, showing a moisture profile as a function of depth and horizontal position, and how it varies with time. Additive water from irrigation or rainfall can be included in the calculation, as well as water loss from evaporation or transpiration. Models for these phenomena are included in the modeling process.

Utilizing measurements of sandy loam soil as a function of time and utilizing a FMCW modulated and demodulated signal we have determined we can identify the depth of the interface between the unsaturated region and the saturated region of soils. Furthermore, we have determined successive measurements of the same region over time yields soil type.

The embodiments of the soil moisture models are derived using successive measurements of a target location (on a farm or greenspace) and knowledge of standard soil conductivity and tension as a function of moisture content for each soil type performed in situ or in the laboratory. The results are then fit to an appropriate functional expression for the conductivity or tension.

What is claimed, is:

1. A remote sensor designed to measure dielectric constant of soil, comprising:
a radio frequency transmitter circuit constructed to transmits an RF signal at a predetermined frequency toward the soil, the transmitted signal defining X and Y boundaries for an imaging area;
a radio frequency receiver circuit constructed to receive from the soil a reflected RF signal responsive to the transmitted RF signal;
a processor configured to calculate soil moisture of a volume of the soil, the volume defined by the imaging area and a soil depth Z, which is inversely proportional to the RF transmission frequency; and
wherein the responsive RF signal is indicative of the dialectic constant of the soil.

2. The remote sensor according to claim 1, wherein the RF transmission frequency is modulated using frequency sweep or code division sweep techniques, which creates function reflected time of arrival vs frequency or code to allow for soil moisture measurement performed at multiple Z depths.

3. A system to determine a comprehensive model of water transport through soil, comprising:
a vehicle having a remote sensor, the remote sensor designed to measure dielectric constant of soil and further comprising:
a radio frequency transmitter circuit constructed to transmits an RF signal at a plurality of predetermined frequencies toward the soil, the transmitted signals defining X and Y boundaries for an imaging area;
a radio frequency receiver circuit constructed to receive from the soil a reflected RF signals responsive to the transmitted RF signals;
a processor configured to calculate soil moisture of a volume of the soil, the volume defined by the imaging area and a soil depth Z, which is inversely proportional to the RF transmission frequency; and
wherein the responsive RF signal is indicative of the dialectic constant of the soil.

4. The system according to claim 3, wherein the vehicle is a plane or aircraft.

5. The system according to claim 3, wherein the vehicle is a land vehicle.

6. The system according to claim 3, wherein the dielectric constant of soil is determined as a function of depth by measuring the energy reflected at the soil air boundary and the unsaturated/saturated region boundary.

7. The system according to claim 6, wherein the vehicle makes multiple passes over time to determine the rate of movement of the unsaturated/saturated region boundary for purposes of determining soil type as a function of depth.

8. The system according to claim 3, wherein soil dielectric measurements are taken for a plurality of imaging areas, each imaging area representing a “pixel” of a target area, the soil measurements being aggregated into a comprehensive model of water transport through soil, pixel by pixel for linear water flow in the z axis and across multiple pixels to develop a three dimensional water flow model.

9. A method to calculate soil moisture, comprising:
receiving successive radar measurements that indicate soil type as a function of soil depth;
applying a transfer function to the measurements that relates soil type to dielectric constant; and
determining soil moisture as a function of depth.

10. The method according to claim 9, further comprising making successive scans or measurements at one location on a farm or field to generate a single dimensional model of water transport through soil in the Z axis.

11. The method according to claim 9, further comprising making successive scans or measurements over a large area to generate a three dimensional model of water transport through soil in the X, Y, and Z axis.

12. The method according to claim 9, further comprising using moisture content from the sensor, and data obtained from the field to improve accuracy via a wireless or wired connection. Data can include wind speed, precipitation, temperature, humidity and other measurements.

13. The method according to claim 9, further comprising using measurements from transponders located inside the soil, or directly on plants, or on irrigation equipment data provide additional information about soil moisture, plant stress, time and quantity of water in active irrigation.

14. The method according to claim 9, further wherein the determining step is heuristic in nature
